(19) United States
(12) Patent Application Publication
(10) Pub. No.: US 2024/0327800 A1
(43) Pub. Date: Oct. 3, 2024

(54) HIGH THROUGHPUT LOCALIZED PASSIVE DIFFUSION DEVICE FOR PATTERNING ORGANOIDS

(71) Applicant: University of Massachusetts, Boston, MA (US)

(72) Inventors: Feiyu Yang, Boston, MA (US); Narciso Pavon, Boston, MA (US); ChangHui Pak, Boston, MA (US); Yubing Sun, Boston, MA (US)

(21) Appl. No.: 18/625,271

(22) Filed: Apr. 3, 2024

Related U.S. Application Data
(60) Provisional application No. 63/493,881, filed on Apr. 3, 2023.

(51) Int. Cl. C12N 5/071; C12M 1/32; C12N 5/00; C12N 5/079

(52) U.S. Cl. CPC C12N 5/0697; C12M 23/12; C12N 5/0062; C12N 5/0618; C12N 2501/41; C12N 2506/00; C12N 2513/00; C12N 2533/54

(57) ABSTRACT

The present disclosure provides a device and methods for patterning organoids. The device includes a plurality of layers vertically arranged. The plurality of layers includes a base layer, a diffusion medium disposed on the base layer, a microwell layer configured above the diffusion medium, and a partitioning layer. The microwell layer includes a lower surface, an upper surface, and one or more microwells. Each microwell defines an opening configured to extend between the microwell and the diffusion medium. The partitioning layer defines a chemical reservoir and one or more medium chambers, where the wall divides the chemical reservoir from the one or more medium chambers.

### CROSS-REFERENCE TO RELATED APPLICATIONS

This application claims the benefit of priority to, and the benefit of, U.S. Provisional Application 63/493,881 filed on Apr. 3, 2023, the disclosure of which is hereby expressly incorporated by reference herein in its entirety.

### STATEMENT REGARDING FEDERALLY SPONSORED RESEARCH

This invention was made with Government Support under Grant No. R21MH130843 awarded by the National Institutes of Health. The Government has certain rights in the invention.

### BACKGROUND

Efforts to understand human brain development, organogenesis, and congenital disabilities are greatly benefited by models that are accessible and closely approximate human brains. Such models may be used not only for human developmental studies but also in drug screening applications. When considering potential brain models, a preferred model is one that is minimally different from human brains in terms of functional mechanisms and physiological activities. One option is to develop neurulation-stage human embryos in vitro. However, according to the most recent International Society for Stem Cell Research (ISSCR) guidelines, although human embryos can be cultured in vitro indefinitely, a rigorous review process is required to limit such experiments to a minimal level for ethical and practical reasons. For at least these reasons, human pluripotent stem cell (“hPSC”)-derived brain organoids present promise as a brain model.

Currently, there are two general methods to derive human brain organoids with hPSCs: spontaneous differentiation and induction differentiation. The first method, spontaneous differentiation, relies on hPSC's intrinsic differentiation capacity to spontaneously organize into cerebral organoids. However, this organization process usually results in heterogeneous and complex morphology. In such cases, individual brain regions are randomly distributed and lack the organized architectures essential for mimicking brain functions. The second method, induction differentiation, introduces external signal factors to induce regional specific organoids. However, no conventional induction method generates a patterned organoid with multiple regional identities.

Deficiencies in the current techniques and methods result in poor approximations of the spatially organized architecture and distinct region-specific identities observed in normal brain development.

In the course of normal brain development, interneurons are generated at the ventral region of the forebrain. These interneurons then tangentially migrate towards the dorsal area of the brain and eventually reach the cortex. Failure to develop according to this pattern may lead to developmental deficiencies.

Furthermore, to induce a gradient-patterned organoid with a synthetic morphogen agonist, the morphogen source should be fixed at one pole of the organoid. Conventionally, organoids are embedded into Matrigel for immobilization. However, Matrigel is subject to batch-to-batch variations due to variations in animal sources, which may cause potentially irreproducible organoid differentiation. Synthetic hydrogels may offer an alternative to Matrigel. However, a readily accessible Matrigel replacement has not yet been developed, and the costs and technical challenges associated with synthetic hydrogels may be prohibitive for large-scale production.

An easy-to-use, high-throughput, and reliable method for developing brain organoids that recapitulates region-specific brain structures with localized differentiation patterning is needed.

### SUMMARY

Disclosed herein are devices and methods for patterning organoids. The device includes a plurality of layers vertically arranged. The plurality of layers includes a base layer, a diffusion medium disposed on the base layer, a microwell layer configured above the diffusion medium, and a partitioning layer. The microwell layer includes a lower surface, an upper surface, and one or more microwells. Each microwell defines an opening configured to extend from the microwell to the diffusion medium. The partitioning layer defines a chemical reservoir and one or more medium chambers. The chemical reservoir includes a wall dividing the chemical reservoir from the one or more medium chambers.

In some aspects, the techniques described herein relate to a device for patterning organoids. The device includes a plurality of layers vertically arranged. The plurality of layers includes: a base layer; a microwell layer; and a portioning layer. The microwell layer disposed above the diffusion medium and includes a lower surface and an upper surface and define one or more microwells. Each of the one or more microwells defines an opening extending between the respective microwell and a diffusion medium; and a partitioning layer disposed above the diffusion medium disposed on the base layer. The partitioning layer includes a wall that defines a chemical reservoir and one or more medium chambers. The wall divides the chemical reservoir from the one or more medium chambers.

In some implementations, cells are disposed within the one or more microwells.

In some implementations, the one or more morphogens are disposed within the chemical reservoir. Diffusion of the one or more morphogens from the lower surface to the upper surface creates a morphogenic gradient within each of the one or more microwells.

In some implementations, the cells include cells of a first type and cells of a second type.

In some implementations, the one or more morphogens differentiate the cells of a first type and the cells of a second type into an organoid disposed within the microwells.

In some implementations, cells of the first type and cells of the second type are differentially disposed along a vertically-oriented differentiation axis according to the morphogenic gradient. The arrangement of the first type and the second type of cells defines a patterning.

In some implementations, the medium chambers are radially outward from the chemical reservoir. In some implementations, each medium chamber contains a microwell.

In some implementations, the microwell layer includes an inorganic polymer.

In some implementations, the diffusion medium includes cross-linked gelatin.

In some implementations, the device further includes one or more alignment structures configured to maintain the relative orientation of the plurality of layers. In some implementations, the one or more alignment structures include a suture.

In some implementations, the device further includes an anchoring layer coupled to the base layer, the anchoring layer defining one or more openings configured to receive the one or more alignment structures, thereby anchoring the one or more alignment structures to the base layer.

In some implementations, the device is sized and configured to fit within a standard six-well plate.

In some aspects, the techniques described herein relate to a method for producing a patterned organoid. The method includes seeding one or more microwells defined in a microwell layer with a plurality of cells to generate a cell aggregate. The method also includes adding one or more morphogens to a chemical reservoir defined by a partitioning layer. The partitioning layer includes a wall that divides the chemical reservoir from one or more medium chambers. One or more microwells are defined within the one or more medium chambers. The method also includes diffusing the one or more morphogens through a diffusion medium disposed beneath the microwell layer. The diffusion medium is in fluid communication with the chemical reservoir. The method also includes releasing the one or more morphogens through openings defined at respective base regions of the one or more microwells. Each respective opening extends between the microwell and the diffusion medium. The method also includes forming a morphogenic gradient within the one or more microwells along a vertically-oriented differentiation axis defined by each of the one or more microwells. The method also includes obtaining a patterned organoid from the cell aggregate included of cells differentiated according to the morphogenic gradient.

In some implementations, the patterned organoid is a neuro-organoid. In some implementations, the one or more morphogens include a sonic hedgehog agonist. In some implementations, the patterned organoid is a dorsal-ventral patterned forebrain organoid. In some implementations, the one or more morphogens include retinoic acid. In some implementations, the patterned organoid is a rostral-caudal patterned hindbrain organoid.

### BRIEF DESCRIPTION OF DRAWINGS

Example features and implementations are disclosed in the accompanying drawings. However, the present disclosure is not limited to the precise arrangements and instrumentalities shown.

FIG. 1 shows an exploded perspective view of a device for patterning organoids according to one implementation.

FIG. 2 shows a cross-sectional side view of the device, according to one implementation. Specifically, FIG. 2 shows a schematic of the diffusion process, wherein morphogenic chemicals added to the device diffuse into microwells containing cells.

FIG. 3 shows a top view of an example of the device assembled, according to one implementation. As shown in the accompanying schematic of a microwell in the microwell layer, an organoid is formed within the microwell. A base region of the microwell is offset form the diffusion layer such that an opening in the microwell extends a distance between the microwell and the diffusion layer.

FIG. 4 shows top views of various layers of the device according to one implementation.

FIG. 5 shows an exploded view of the device, according to another implementation.

FIG. 6 shows a schematic illustrating an example method of forming a patterned organoid using the device, according to one implementation.

FIG. 7 shows a perspective view of an example device, according to one implementation.

FIG. 8 illustrates an example method of generating a human cerebral organoid using the device. Panel A shows a schematic of the brain organoid generation protocol. Panel B shows a phase contrast image of representative embryoid bodies (“EBs”) on day 1, day 3, and day 8, having a scale bar of 10 mm. Panel C is a photograph of an example device having EBs grown within. Panel D shows a photograph of organoids placed in a cryomold along with sutures.

FIG. 9 shows a schematic example of a method of fabricating the microwell layer of the device, according to one implementation.

FIGS. 10A-10B show top view a side views of a microwell template used in the fabrication method of FIG. 9.

FIG. 11 shows images of the generation of dorsal-ventral patterning of a human forebrain organoid patterned using an example device used with a gradient of sonic hedgehog (“Shh”) agonist as a morphogen. Panel A shows a protocol for the human forebrain organoid dorsal-ventral patterning, according to one implementation. Panel B shows immunostaining of key markers for dorsal-ventral patterning of forebrain organoids under various conditions, including a group receiving a gradient of purmorphamine, a group receiving uniform purmorphamine supplemented in the medium chamber and morphogen chamber, and a group receiving no purmorphamine supplemented in the medium chamber and morphogen chamber.

FIG. 12 shows images of active neurogenesis and astrogenesis of dorsal-ventral patterned forebrain organoids, generated using an example device, illustrated by immunostaining key markers from day 85 to day 186.

FIG. 13 shows a quantification of the polarity of organoids generated using an example of the device, using imaging analysis. Panel A shows the original Nkx2.1 staining image. Panel B illustrates use of the Sobel edge detector function to quantify the changes in intensity across pixels in a 3×3 kernel, expressed partly through the display of vectors. The sum of these vectors gives a specific gradient direction for each pixel in the image. Panel C shows the mean pixel gradient of the vectors of Panel B, giving the total gradient in the organoids.

FIG. 14 shows images of the generation of rostral-caudal patterning of a human brain organoid using a gradient of retinoic acid (“RA”). Panel A shows a protocol for the brain organoid rostral-caudal patterning, according to one implementation. Panel B shows immunostaining key markers for rostral-caudal patterning of human brain organoids under various conditions, including a group receiving a gradient of RA, a group receiving uniform RA supplemented in the medium chamber and morphogen chamber, and a group receiving no RA supplemented in the medium chamber and morphogen chamber.

### DETAILED DESCRIPTION

Provided herein are devices, systems, and methods disclosed herein provide for a device for patterning organoids. As used herein, the device may also be referred to as a bioreactor or a gradient generation device.

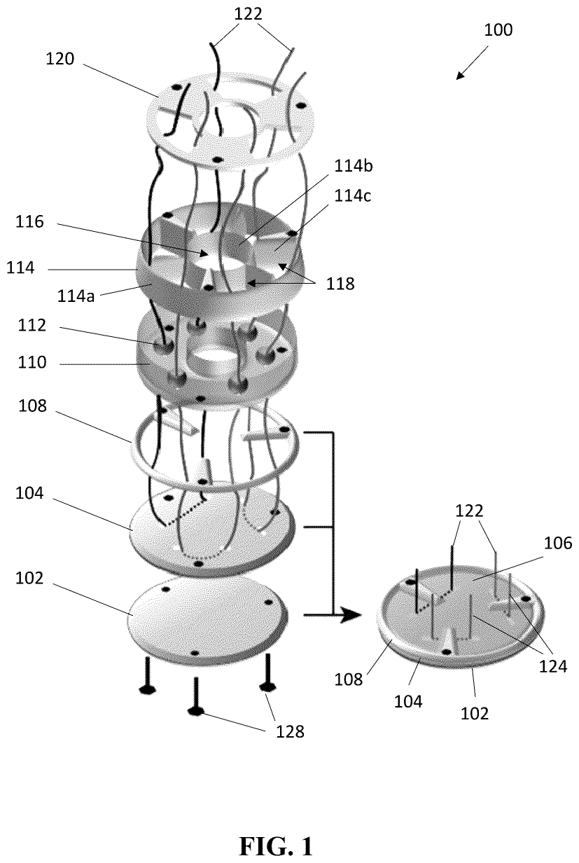

Referring generally to the figures, FIG. 1 shows a device 100 for patterning organoids according to various implementations. As shown in FIG. 1, the device 100 includes a plurality of layers vertically arranged. Specifically, the plurality of layers are aligned along a common vertical axis. In some aspects, arranging the layers in a vertical configuration promotes directionally controlled diffusion of added small molecules, such as morphogens, through the influence of gravity. Accordingly, as used herein “vertical” means parallel to the prevailing direction of gravity.

In the implementation shown in FIG. 1, the plurality of layers includes a base layer 102. As shown, the base layer 102 is positioned at the bottom of the device 100 and comprises a solid or substantially solid surface to prevent leakage of the contents of the device 100. As shown in FIG. 1, the device 100 further includes an anchoring layer 104 disposed above the base layer 102. The anchoring layer 104 is further described below.

As shown in FIG. 1, a diffusion medium 106 is disposed on the anchoring layer 104. The diffusion medium 106 is characterized by its porosity, which may be adjusted to control the release rate of small molecules through the diffusion medium 106. In the illustrated implementations, the diffusion medium 106 is a crosslinked gelatin. Accordingly, the diffusion medium 106 may also be referred to as a “gelatin layer” or a “cross-linked gelatin”. In other implementations, the diffusion medium 106 may be selected from any material that may be configured to control the release rate of small molecules. In other implementations described herein, the anchoring layer 104 may be omitted. In such implementations, the diffusion medium 106 may be disposed on the base layer 102.

As shown, the device 100 further includes a diffusion medium containment layer 108. In the illustrated implementation, the diffusion medium containment layer 108 includes a ring extending circumferentially around a perimeter of the diffusion medium 106 and is configured to confine the diffusion medium. As shown in FIG. 1, the medium containment layer 108 includes a plurality of projections extending radially inward from a raised circumferential edge of the medium containment layer 108.

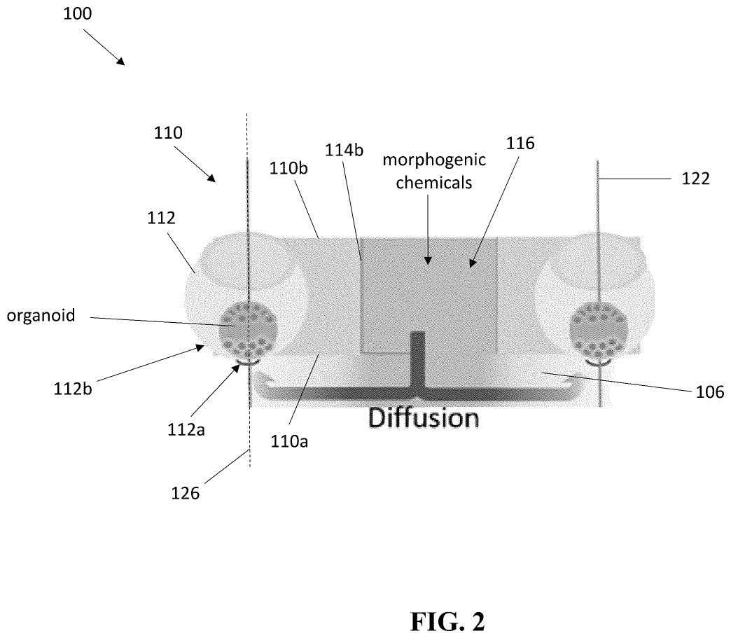

The device 100 further includes a microwell layer 110 disposed above the diffusion medium 106. As shown, for example, in FIGS. 1 and 2, the microwell layer 110 includes a lower surface 110a, an upper surface 110b, and one or more microwells 112. As shown in FIG. 1, the microwell layer 110 is annular shaped. In some implementations, the microwell layer 110 may be formed from an inorganic polymer. For example, in the illustrated implementations, the inorganic polymer is polydimethylsiloxane (“PDMS”). In some implementations, the microwell layer 110 is a PDMS microwell sheet.

As shown in FIG. 1 the microwell layer 110 includes six microwells 112. In other implementations, the microwell layer 110 may include more or less microwells 112. As shown in FIG. 2, In the illustrated implementation, the microwells 112 are spherical. However, in other implementations, the microwells 112 may be other suitable shapes, such as cylinders, for example. each microwell 112 defines an opening 112a that extends from the microwell 112 to the diffusion medium 108. Furthermore, each microwell 112 has a base region 112b. In some implementations, as shown in FIG. 2, the base region is in direct contact with the diffusion medium 106. However, in other implementations, the base region 112b is offset from the diffusion medium 106 by a distance.

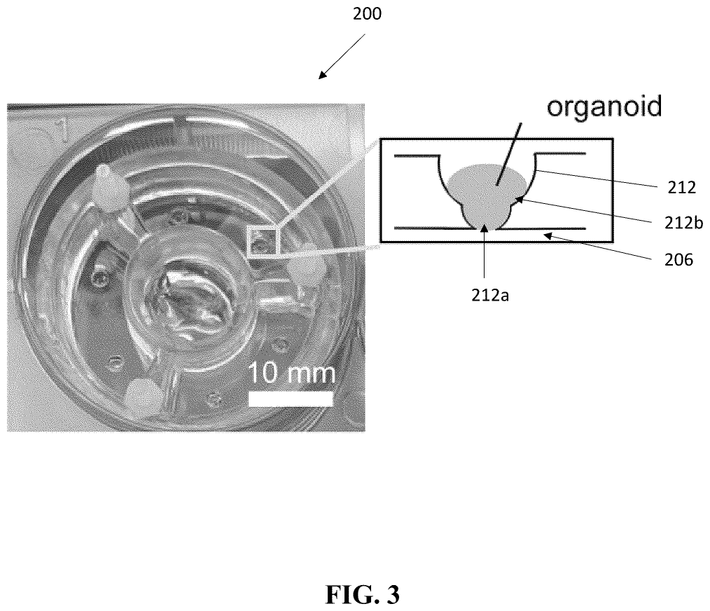

For example, FIG. 3 shows a device 200 similar to device 100. Accordingly, like components are identified using like reference numbers. However, in device 200, the base region 212b of the microwell 212 is offset from the diffusion medium 206 by a distance and the opening 212a extends from the base region 212b to the diffusion medium 206.

As shown in FIG. 1, a partitioning layer 114 is positioned above the microwell layer 110. The partitioning layer 114 includes an outer wall 114a that extends circumferentially around a perimeter of the microwell layer 110 when the device 100 is assembled. Accordingly, the outer wall 114a of the partitioning layer 114 is configured to confine the outer perimeter of the microwell layer 110. As shown, the partitioning layer 114 also comprises an inner wall 114b that defines a chemical reservoir 116 within an interior region of the partitioning layer 114. As shown, the chemical reservoir 116 is in fluid communication with the diffusion medium 106. However, in another implementation, the one or more microwells are positioned centrally within the device and are surrounded by the chemical reservoir.

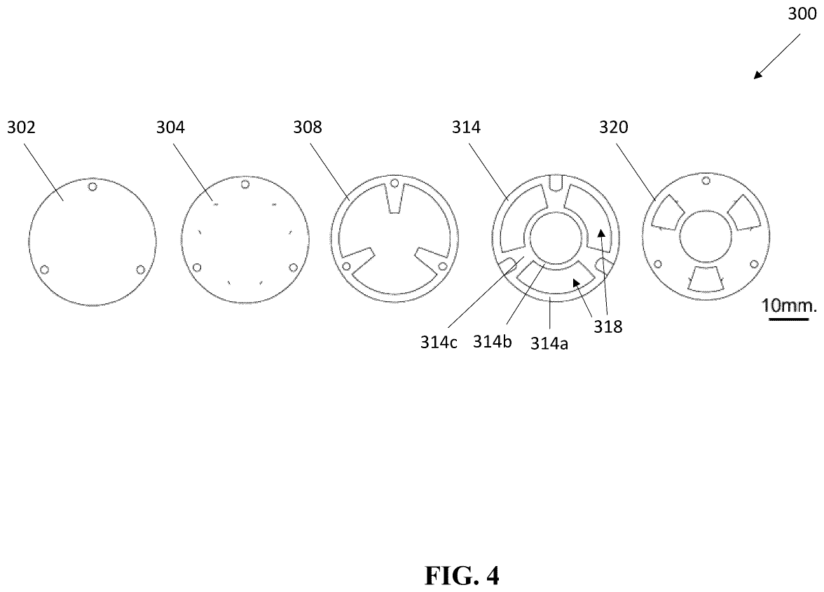

Furthermore, the partitioning layer 114 also defines one or more medium chambers 118 distributed radially about the chemical reservoir 116. Specifically, the medium chambers 118 are defined between the inner wall 114b and the outer wall 114a such that the medium chambers 118 are radially outward relative to the chemical reservoir 116. Thus, the inner wall 114b that defines the chemical reservoir 116 divides the chemical reservoir 116 from the one or more medium chambers 118. As shown, chamber dividing walls 114c separate adjacent medium chambers 118 from one another. For example, the illustrated embodiment in FIG. 1 includes six medium chambers 118 defined between six chamber dividing walls 114c. However, other implementations may include more or less medium chambers 118 and chamber dividing walls 114c. For example, FIG. 4 shows layers of a device 300 similar to device 100. Accordingly, like components are identified using like reference numbers. For example, the device 300 includes: a base layer 302 positioned at the bottom of the device 300; an anchoring layer 304 disposed above the base layer 302; a diffusion medium containment layer 308; a partitioning layer 314 disposed above the anchoring layer 304 and having an inner wall 314b that defines a chemical reservoir and divides the chemical reservoir from medium chambers 318, an outer wall 314a, and chamber dividing walls 314c that separate adjacent medium chambers 318; and an end layer 320. However, the partitioning layer 314 of device 300 defines three medium chambers 318.

Furthermore, as shown in FIG. 1, each medium chamber 118 contains a single microwell 112. As such, the chamber dividing walls 114c prevent the contents in or around one microwell 112 from interacting with the contents in or around an adjacent microwell 112. In other implementations, each medium chamber 118 may contain more than one microwell 112. In the illustrated implementations, the chemical reservoir 116 is cylindrically-shaped and defines a vertically-oriented reservoir axis. As shown, the one or more microwells 112 are positioned radially about the reservoir axis of the chemical reservoir 116.

Additionally, FIG. 1 shows that device 100 also includes an end layer 120 disposed above the partitioning layer 114. As shown in FIG. 1, the end layer 120 defines a plurality of openings. Specifically, end layer 120 defines three openings. As shown, the openings of the end layer 120 may be rotationally offset relative to the medium chambers 118 defined in the partitioning layer 114 below. Furthermore, in other implementations, the end layer 120 may include more or less openings. For example, the end layer 120 may be substantially solid so as to fully seal the partitioning layer 114. In other implementations, the end layer 120 may include only a central opening sized and configured to align with the chemical reservoir 116 to permit the addition of chemicals to the chemical reservoir 116.

In the implementations illustrated in FIG. 1, the base layer 102, the anchoring layer 104, the diffusion medium retention layer 108, the partitioning layer 114, and the end layer 120 are acrylic. However, in other implementations, any suitable material may be used to fabricate the various layers. For example, in some implementations, the layers 102, 104, 108, 114, and/or 120 are formed from another biocompatible material such as polystyrene. In use, the device 100 is formed by coupling the layers together. In some implementations, the device 100 is cylindrically-shaped when the layers are coupled together. For example, the device 100 shown in the illustrated implementations is sized and configured to fit within a standard six-well plate. In some implementations, the base layer 102 may include holes for use in assembling the device 100. For example, in FIG. 1, the base layer 102 has three threaded holes configured to receive respective fixing members used to couple layers 102, 104, 108, 114, and 120 to each other. In the illustrated implementation, the fixing members are nylon screws. In other implementations, other mechanisms, such as adhesives or laches may be used to secure the various layers of the device relative to each other.

As shown, for example, in FIG. 2, cells are disposed within the microwells 112. In the illustrated implementations, the cells are stem cells. Specifically, the cells shown are human pluripotent stem cells. In some implementations, the human pluripotent stem cells may be human embryonic stem cells (“hESCs”). In other implementations, the cells may be human induced pluripotent stem cells (iPSCs). In other implementations, the cells may be derived from other organisms. In some implementations, the type of cell disposed within the microwells may be selected based on an intended organoid to be generated.

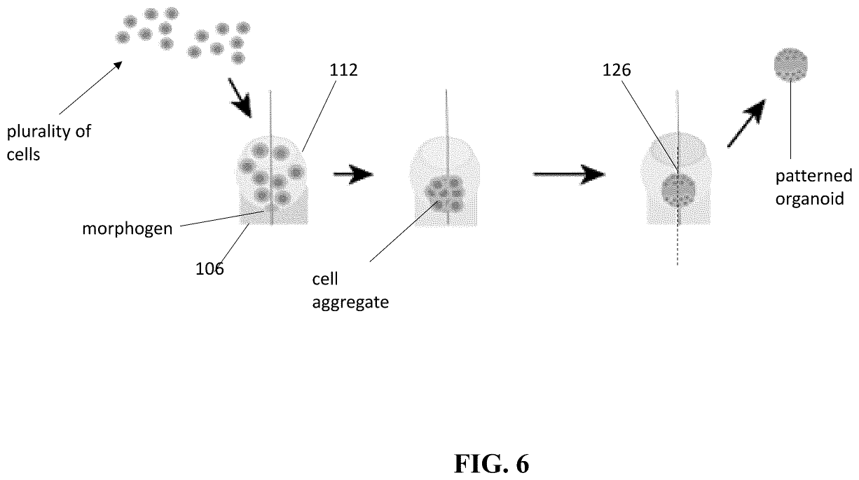

In other implementations, the cells may be LGR5+ intestinal stem cells, which may be used to generate intestine organoids. Use of stem cell types in cooperation with the device is beneficial given that such cells have the capacity to differentiate into other cell types. As shown, for example, in FIG. 2, a quantity of cells is added to each microwell 112 so as to form a cell aggregate. Another example of cells forming a cell aggregate is shown in FIG. 6.

In some aspects, in addition or in the alternative to the preceding aspects, chemicals may be disposed within the chemical reservoir 116. As shown in FIG. 2, when chemicals are added to the chemical reservoir 116, some of the chemicals travel into the diffusion medium 106 disposed below the chemical reservoir 116. For example, in some implementations, the chemicals may be biomolecules. For example, in some implementations, the biomolecules include one or more morphogens. In the implementation illustrated, for example, in FIG. 2, the chemicals are morphogens including purmorphamine as a sonic hedgehog (“Shh”) agonist and retinoic acid (“RA”). In some implementations, a different Shh agonist may be selected. For example, in some implementations, the Shh agonist is SAG (Smoothened Agonist). Furthermore, in some implementations, another rostral-caudal morphogen may be used. For example, in some implementations, the rostral-caudal morphogen is a Wnt signaling regulator, including agonist (e.g., CHIR99021 or 6-Bromoindirubin-3′-oxime) or antagonist (e.g., IWP2). Additional rostral-caudal factors include Fibroblast Growth Factors (FGF), such as the FGF8 subfamily (Fgf8, Fgf-17, and FGF-18).

As shown in the illustrated implementations, the inner wall 114b of the partitioning layer 114 prevents direct flow of chemicals from the chemical reservoir 116 into the one or more medium chambers 118 and/or from the chemical reservoir 116 into the one or more microwells 112. Therefore, contact between the chemicals added to the chemical reservoir 116 and the microwells 112 is mediated by the diffusion medium 106. In some implementations, the chemicals, such as one or more morphogens, diffuse through the diffusion medium 106 upwards toward the microwell layer 110. As shown in FIG. 2, morphogens diffused through the diffusion medium 106 enter the opening 112a of a respective microwell 112 and travel through the microwell 112 from the lower surface 110a of the microwell layer 110 to the upper surface 110b of the microwell layer. The pattern of morphogen diffusion through the microwell 112 creates a morphogenic gradient within the microwell 112. The size of the openings 112a may be controlled to regulate the rate of diffusion of the one or more morphogens into the microwell 112.

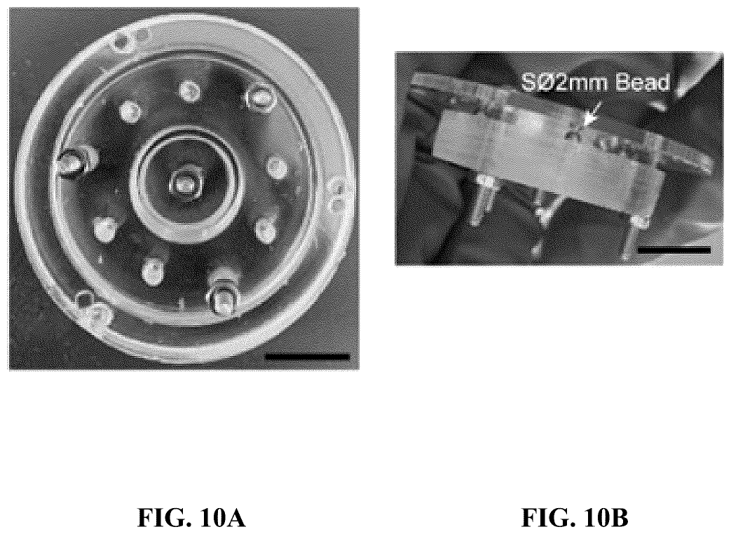

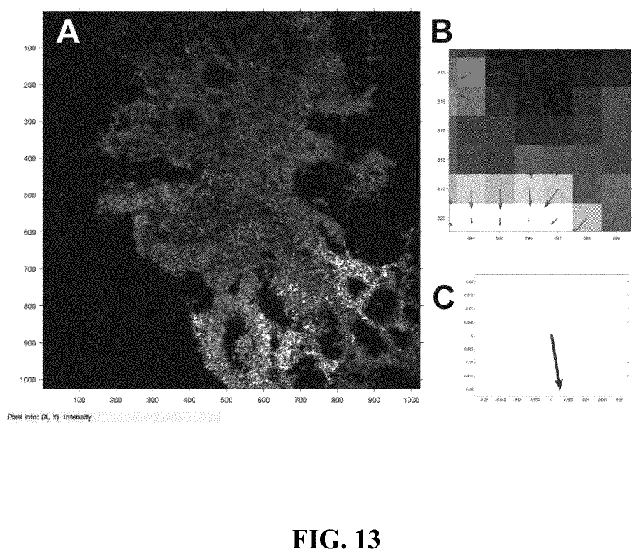

In the illustrated implementations, contact between the cells in the microwell 112 and the one or more morphogens causes the cells to differentiate. Furthermore, some cells in the cell aggregate are exposed to higher amounts of morphogens than others in accordance to relative amounts of morphogen exposure. Thus, in the illustrated implementations, the microwells 112 contain cells of a first cell type and cells of a second cell type. As the cell aggregate differentiates according to the particular morphogens it is exposed to, the cell aggregate becomes an organoid. In the implementation shown in FIG. 2, the organoid is a neuro-organoid. In the illustrated implementation, the organoid was exposed a morphogenic gradient where the morphogen was a Shh agonist, causing the patterned organoid to become a dorsal-ventral patterned forebrain neuro-organoid. An additional example of a dorsal-ventral patterned forebrain organoid is provided herein in FIGS. 10-12. Furthermore, in other implementations provided herein, the organoid was exposed to a morphogenic gradient where the morphogen was RA, causing the patterned organoid to become a rostral-caudal patterned hindbrain neuro-organoid. An example rostral-caudal patterned hindbrain organoid is provided herein in FIG. 13. In other implementations, the organoid is a different organoid, for example, a cardiac organoid.

In some implementations, the chemical reservoir 116 may be partitioned so as to form multiple chemical reservoirs 116, wherein each partition of the chemical reservoir is cable of containing a morphogen different from the other partition. Furthermore, the diffusion medium containment layer 108 may also include partitions so as to form regions of diffusion medium that are fully or substantially partitioned. In these alternate implementations, different microwells 112 within a single microwell layer 110 may be exposed to different morphemic gradients and thus may be differentiated into different organoids. For example, organoids in some microwells 112 may be exposed to a Shh agonist while other organoids in other microwells 112 are exposed to RA. Thus, a single microwell layer 110 may be used to produce both dorsal-ventral and rostral-caudal patterned organoids.

As shown, for example, in FIG. 2, cells of the first type and cells of the second type are differentially disposed along a vertically-oriented differentiation axis defined by a direction of the morphogenic gradient, wherein the arrangement of the first type and second types of cells defines a patterning. As used herein, “differentially disposed” refers to the first and second types of cells having different relative distributions along the differentiation axis 126. In some implementations, chemicals may be added to the medium chambers to assist or direct cell differentiation or to nourish the cells or otherwise contribute to sustaining the cells.

In some aspects, in addition or in the alternative to the preceding aspects, the device further includes one or more alignment structures 122 configured to maintain the relative orientation of the plurality of layers. The device shown in FIG. 1, for example, includes a plurality of alignment structures 122 in the form of elongated members extending between the anchoring layer 104 and the end layer 120. Specifically, the elongated members 122 in the illustrated implementation are sutures. In the illustrated implementation, the sutures are nylon sutures. However, elongated members as alignment structures 122 may formed from any other suitable material. As shown, the anchoring layer 104 comprises a plurality of holes configured to receive the alignment structures 122 and anchor the alignment structures 122 to the anchoring layer 104, which is itself coupled to the base layer 102. These holes are placed such that they allow coupling with other layers of the device 100 but do not create pathways for potential leakage of the diffusion medium 108. As shown, the plurality of holes in the anchoring layer 104 are aligned with the openings 112a of respective microwells 112 such that alignment structures 122 are able to pass through respective vertically aligned holes and openings 112a such that they are parallel to the reservoir axis.

As shown, the sutures as alignment structures 122 pass through the diffusion medium 106 and microwells 112. In some implementations, the alignment structures 122 are non-absorbable sutures so as to resist cell attachment. In addition to controlling the relative orientation of the plurality of layers, the alignment structures 122 immobilize the organoids, thereby guiding organoid development. In some implementations, Matrigel may be used to fix the organoids.

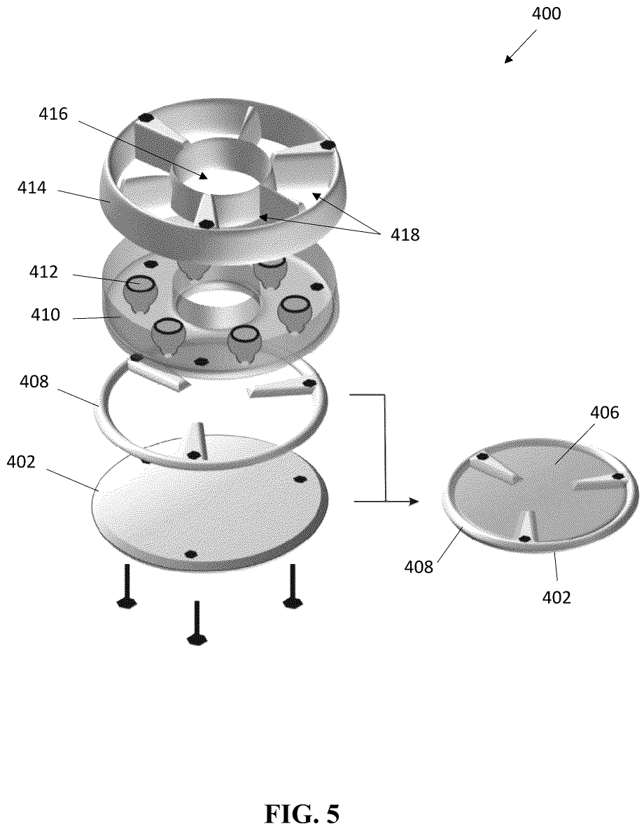

In other implementations, the alignment structures 122 are absent. For example, FIG. 5 shows a device 400 similar to device 100. Accordingly, like components are identified using like reference numbers. However, the device 400 does not include suture-like alignment structures 122. Accordingly, the device 400 does not contain an anchoring layer 104. Thus, as shown, the diffusion medium 406 is disposed directly on the base layer 402. For example, the device 400 includes: a base layer 402 positioned at the bottom of the device 400; a diffusion medium 406 disposed on the base layer 402; a diffusion medium containment layer 408 disposed above the base layer 402; a microwell layer 410 disposed above the diffusion medium 406 and including microwells 412; and a partitioning layer 414 positioned above the microwell layer 410 that defines a chemical reservoir 416 and medium chambers 418.

Furthermore, the device shown in FIG. 5 is also assembled without an end layer 120. The present disclosure, in one aspect, provides for a method of producing a patterned organoid. As shown in FIG. 6, for example, the method includes: seeding the one or more microwells 112 with a plurality of cells to generate a cell aggregate; adding one or more morphogens to the chemical reservoir 116 (chemical reservoir 116 not explicitly shown in FIG. 6 but is configured according to the device 100 as shown in FIG. 1); diffusing the one or more morphogens through the diffusion medium 106; releasing the one or more morphogens through an opening 112a defined by one or more microwells 112 defined in a microwell layer 110; forming a morphogenic gradient within the one or more microwells 112 along a vertically-oriented differentiation axis 126; and obtaining a patterned organoid from the cell aggregate included of cells differentiated according to the morphogenic gradient.

In one aspect, disclosed herein is a high-throughput, reliable, control-release diffusion device to pattern hPSC-derived organoids into dorsal-ventral (“D-V”) and/or rostral-caudal (“R-C”) patternings.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### Examples

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

### Example 1. Fabricating and Calibrating a Gradient Generation Device

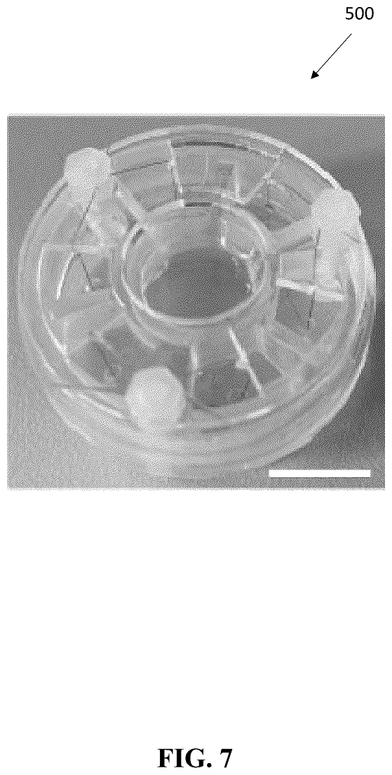

FIG. 7 shows an example device 500 similar to device 100. Accordingly, like components are identified using like reference numbers. As shown, the device 500 includes five layers of acrylic structures, one microwell layer (a ring-shaped PDMS microwell sheet also referred to as a “PDMS layer”), three sections of alignment structures (nylon surgical sutures), and one layer diffusion medium (crosslinked gelatin). In the PDMS layer, six microwells were arranged at equal distances from the chemical reservoir defined in the center of the annular-shaped device 500. Openings on the bottom of each microwell were created (Ø 200 μm), allowing the surgical sutures to thread through. The surgical sutures allowed organoids to be cultured in a Matrigel-free medium and fixed to the device 500. In practice, the cells grew around the 5-0 surgical suture section. The diffusion layer included 15% gelatin crosslinked with 50 mg/ml transglutaminase for the controlled release of the perspective small molecules and sealed the openings underneath each respective microwell. LA partitioning layer was mounted onto the PDMS layer, partitioning the microwell layer into seven sections, creating six separate medium chambers for six microwells and one central chemical reservoir for receiving and holding a morphogen. The chemical reservoir was configured to be loaded with morphogen such that, once loaded into the chemical reservoir, the morphogen would diffuse through the diffusion layer and reach one pole of an embryoid body (“EB”) immobilized within a microwell.

The 5-0 monofilament nylon surgical suture was fixed to be vertically oriented by securing the surgical suture between the anchoring layer and end layer. The suture was formed from a smooth, non-absorbable material that resists cell attachment. Thus, subsequently generated EB are not exposed to external factors (such as Matrigel) that could affect growth and differentiation. The base layer was positioned at the bottom of the device 500 to prevent leaking. The device 500 was assembled and fixed with nylon M2 screws. The partitioning layer and other acrylic structures were made with 6.35 mm and 1.5 mm thickness acrylic sheets, respectively.

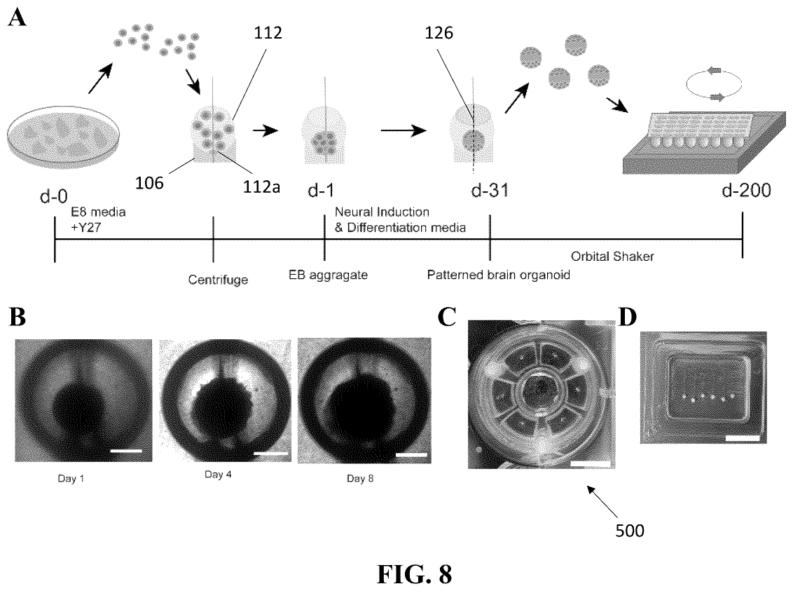

In the example shown in FIGS. 7, the device 500 was designed as an annular ring shape, with the middle chamber containing the morphogen and the surrounding chambers for containing cells in medium chambers. Small openings were created at the bottom of each microwell to allow cells therein to be in contact with the diffusion layer positioned beneath, as shown in FIG. 8. As shown in FIG. 8, prospective small molecules from the morphogen chamber diffused through the diffusion medium and were control-released through the openings into the bottom side of organoids grown in the microwell.

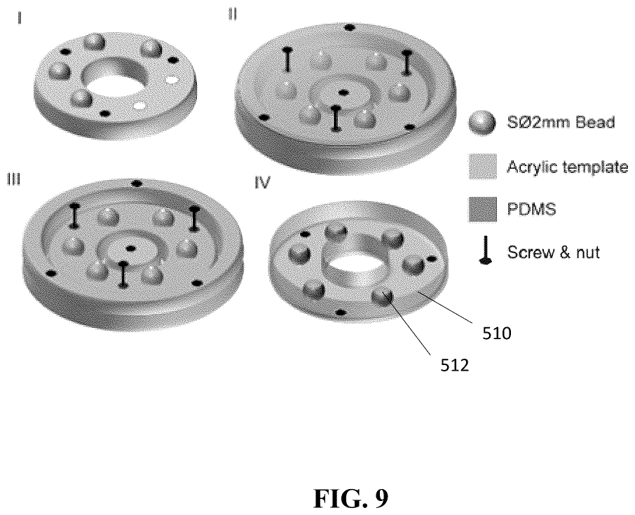

As shown in FIGS. 9 and 10A-10B, the microwell layer was fabricated with PDMS and individual spherical microwells were fabricated using spherical beads (SØ 2 mm) and acrylic templates. As shown in FIG. 9, 1.5 mm diameter holes were laser-printed on the 3.75 mm thick acrylic sheet to create a holding template containing holding spots for the beads. The beads were placed into the holding template using tweezers. Then, as shown in FIGS. 10A and 10B, a 1.5 mm thick acrylic lid was screwed on top of the beads. To generate the integrated inner and outer walls for the microwell layer, ring-shaped and cylinder-shaped acrylic slabs were screwed onto the acrylic lid, leaving gaps in between the templates to generate PDMS walls in the microwell layer. The 10:1 ratio degassed PDMS was poured into the template. Due to the tight contact between the beads and the acrylic lid, a custom-designed hold in the bottom center of the microwell layer was created. The PDMS microwell layer was placed in a 4 C.° fridge overnight to remove the air bubbles and was then placed in a 65 C.° oven overnight to complete crosslinking.

The PDMS microwell layer, nylon M2 screws, and nylon M2 nuts were then autoclaved for sterilization. The acrylic layers of the device 500 were UV-ozone sterilized for 10 minutes on each side. The device 500 was then assembled inside a biosafety cabinet.

Before cell seeding, the device 500 was incubated in 2% pluronic at room temperature for two hours, then rinsed three times with deionized water, then incubated in deionized water overnight at a 4-degree refrigerator. Then the device 500 was blown dry in preparation for adding gelatin as the diffusion medium. The diffusion medium was made after the device 500 was assembled and one day before cell seeding. Gelatin and transglutaminase were dissolved with deionized water and sterilized by autoclaving or filtering. During the experiment, the gelatin was placed into a 37 C° water bath for 1 hour to liquefy. 800 μl of 18.75% gelatin was mixed with 200 μl of 50 mg/ml transglutaminase for crosslinking, making the final concentration 15%. The diffusion medium containment layer was partitioned into three sections. 300 μl of the mixture solution was pipetted slowly to fill each section of the diffusion medium containment layer, then another 300 μl was added into the middle region of the diffusion medium containment layer. The device 500 was placed into a 35 C.° incubator overnight for the full crosslinking, then 25 mins into a 65 C.° oven to destroy enzymes.

### Example 2. High Throughput Localized Passive Diffusion Device for Patterning Dorsal-Ventral Forebrain Neuro-Organoids

### Cell Culturing

As shown in FIG. 8, hESCs (H9) were maintained using E8 medium on the 1% geltrex coated 60 mm petri dishes. The cells were sub-passaged every 3 to 4 days by 80% to 90% confluence. Before cell culturing, the device 500 was incubated in 2% pluronic for one hour, rinsed with 1×DPBS three times, and dried out before adding the gelatin-transglutaminase mixture to layer 3. Before cell seeding, the medium and medium chambers were incubated at room temperature with E8 basal for up to 24 hrs. The gelatin in the microwells was aspirated after being soaked in the E8 basal before cell seeding.

Cells were seeded using E8 medium supplemented with 10 uM Rock inhibitor (Y27). 100, 000 cells suspended in 5 μl medium were then pipetted into each microwell. Then, the device 500 was centrifuged at 200 g for 2 mins. Then, another 1 ml and 500 μl of medium were added into the medium chamber and the morphogen chamber, respectively. The device 500 was placed into a 37 C.° incubator overnight to generate a cell aggregate. As shown In FIG. 8, embryoid bodies were generated in each microwell around the suture at the bottom of the microwell the next day (day 1). It was observed that the EBs were immobilized by growing them around the sutures. To grow EBs around the sutures, a high density of hESCs was seeded directly into the microwell. The size of the EB spheroid at day 8, around 500 μm, was consistent with the diameter of the EB at day 1. The size of EB spheroid could be grown by changing the E8 medium and adding 1% PS daily during continued culturing.

Induction of Dorsal-Ventral Patterned Forebrain Organoids with a Gradient of Shh

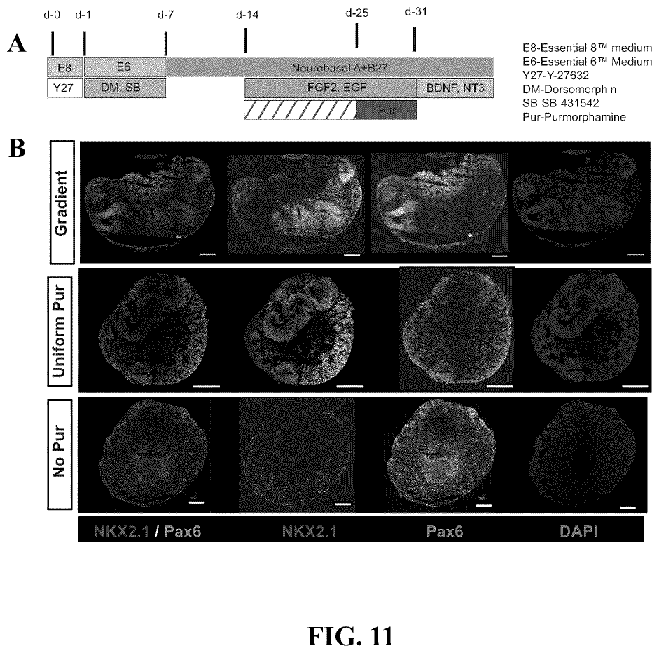

As shown in FIG. 11, for differentiation of the EB spheroid into D-V patterning, the neural induction was started at day 0 when the EBs were first generated. Essential 6 (E6) medium supplemented with 1% PS, 5 μM dorsomorphin (DM), and 10 μM SB-431542 for both medium and morphogen chambers for six days for dual SMAD inhibition. As shown in FIG. 11, from day 7 to day 30, neurobasal medium (NM) was used supplemented with 2% B27, 1% Glutamax, 1% PS, 20 ng/ml EGE, and 20 ng/ml FGF-2 as the basal. To generate a D-V patterned organoid, 5 μM of purmorphamine and 5 μM of IWP-2 were supplemented to the basal medium in the morphogen chamber for five days between day 14 to day 30. To generate cortical organoids, the basal medium was used for both the medium and the morphogen chamber. To generate subpallial organoids, purmorphamine and IWP-2 supplement medium were used for both chambers. The medium was changed daily until day 30.

### Cryopreservation and Sectioning

The organoids were then rinsed with 1×DPBS two times for 10 mins, then fixed by incubating in 4% paraformaldehyde (Electron Microscopy Sciences) at 4 C.° overnight. Then, the organoid was rinsed three times with 1×DPBS and incubated in 30% sucrose/DPBS solution for 48 to 72 hours in the 4 C°. Then, the organoid was released from the device 500 along with the suture and moved into O.C.T compound in a 25×100 mm Cyromold. Then, the sample was quick-frozen into a cryosectioning block with 100% ethanol/dry ice slurry. The block could be stored long-term in −80 C°. Before cryosectioning, the sample was defrosted at −25 C.° for 30 minutes. The organoid was sectioned into 12 to 25 μm thickness slices and picked up into microscopic slices. The slices were preserved at −80 C°.

### Immunostaining

After differentiation, the organoids were released from the device 500 for future sectioning and immunostaining along with the suture. The suture provided a directionality reference for data analysis. For immunostaining, the sections were washed with 0.2% Triton (Fisher Scientific) diluted in 1×DPBS two times for 15 mins to rinse off the OCT compound. Then, the organoid sections were circled in the immunostaining area with a PAP. Then, the cells were incubated in 10% goat/donkey serum for 2 hours at room temperature. Then, the section was incubated in primary antibodies diluted in 10% goat/donkey serum in a humidified chamber at 4° C. overnight. After binding of primary antibodies, the sample was rinsed with 1×DPBS two times for 15 minutes, then incubated in secondary antibodies and DAPI for 2 hours at room temperature, and then rinsed for another two times. The sample was then fixed in Fluoromount with cover slides for preservation and imaging.

Antigen retrieval is required for some primary antibodies before the blocking step. The Antigen retrieval buffer is made by mixing 2.94% of Tri-sodium citrate and 0.5% of Tween 20 into deionized water. The buffer was adjusted to a pH of 6 before use. The slices were submerged into antigen retrieval buffer in a heat-resistant Coplin staining jar, then steamed for 20 mins in a food steamer. Then the slices were incubated in 0.2% Triton for another 15 mins and proceeded to the blocking step.

Primary antibodies used include: mouse anti-Pax6 (1:500 BD Biosciences, 561462), rabbit anti-NKX2.1 (1:500, Abcam, Ab76013), rat anti-CTIP2 (1:2000, Abcam ab18465), rabbit anti-TBR2 (1:1000, Abcam ab23345), mouse anti-SATB2 (1:1000 Abcam ab51502), rabbit anti-NEUN (1:1000, EMD Millipore ABN78), rabbit anti-S100B (1:1000, Sigma S2644), chicken anti-MAP2 (1:5000, Abcam ab5392), mouse anti-SYNAPSIN (1:500, Synaptic System 111011), goat anti-OTX2 (1:100, R&D Systems), rat anti-HoxB4 (1:20, DSHB).

Secondary antibodies used include goat ant-rabbit IgG-Alexa Fluor 555 (1:500, Life Technologies), goat anti-mouse IgG-Alexa Fluor 488 (1:500, Life Technologies), goat anti-mouse IgG-Alexa Fluor 647 (1:500, Life Technologies), goat anti-chicken IgG-Alexa Fluor 647 (1:500, Life Technologies), donkey-anti goat IgG-Alexa Fluor 647 (1:1000, Life Technologies), donkey-anti rat IgG-Alexa Fluor 488 (1:1000, Life Technologies), donkey-anti rabbit IgG-Alexa Fluor 555 (1:1000, Life Technologies).

### Microscopy

An inverted epifluorescence microscope (Leica Dmi8; Leica Microsystems) was used to collect fluorescent images. Confocal images were collected with NIS-Elements AR software using Nikon A1 Resonant scanning confocal inverted microscope. ImageJ was used for processing images.

A gradient of synthetic Shh agonist (purmorphamine) was introduced during the organoid differentiation process. Dorsal-ventral patterning was established on the forebrain organoid. Specifically, a morphogenic gradient was generated by positioning the organoid in the microwell such that only one side of the organoid was in direct contact with the diffusion medium. As shown in FIG. 11, by adding 5 μM of purmorphamine for a continuous of five days between day 14 to day 30, a higher expression level of NKX2.1 makers was established at the bottom half of the organoid, indicating ventral fate differentiation. At the top half of the organoid, a dominant expression of PAX6 was observed, indicating dorsal fate differentiation. In control groups, purmorphamine (Uniform Pur) or without purmorphamine (No Pur) was added in the medium chamber and the morphogen chamber. No D-V patterning was established due to the lack of gradient. After patterning, the organoids were released from the device 500 and transferred to a low attachment 96 well plate on an orbital shaker for long-term culturing.

### Long-Term Culturing Reveals the Normal Development of the Suture Integrated Brain Organoid

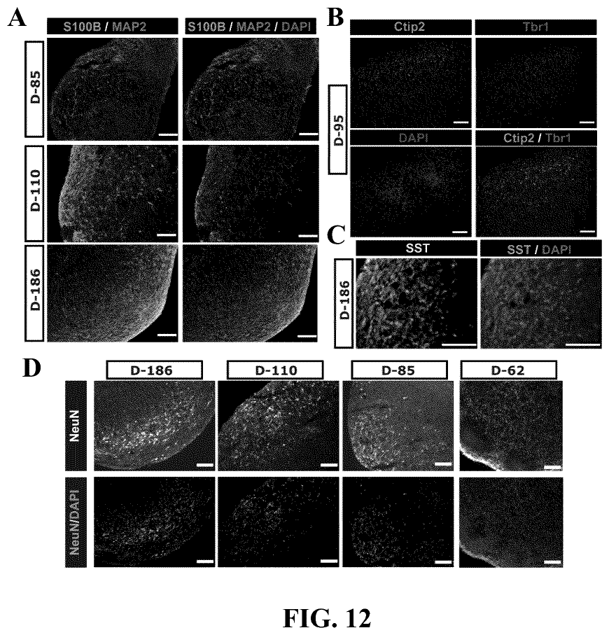

The D-V patterned organoids were cultured over a long period to generate late-stage neurons. Pax6 and NKX2.1 expressing cells are generally present at the early stage of neural induction (<50 days). As shown in FIG. 12, as the organoid matures, late-stage neurons start to appear, indicating normal brain organoid development. The presence of S100B+ developing astrocytes was abundant presents at day 85. The mature neuron NeuN+ was beginning to present at day 65. At day 95, cortical neurons CTip2+ and Tbr1+ were reliably detected at the outside layers of the organoid. At day 186, interneuron marker SST is detected. The percentage of MAP2+ dendrites was significantly increasing from day 85 to day 186. Late-stage neurons and astrocytes indicate normal neurogenesis and astrogenesis of the D-V patterned forebrain organoid. Furthermore, as shown in FIG. 13, the polarity of the organoids was quantified using imagining analysis. Panel A in FIG. 13 shows the original Nkx2.1 staining image. As shown in Panel B, a Sobel edge detector function was used to quantify the changes in intensity across pixels in a 3×3 kernel, expressed partly through the display of vectors. The sum of these vectors provided a specific gradient direction for each pixel in the image. Panel C shows the mean pixel gradient of the vectors of Panel B, giving the total gradient in the organoids.

### Example 3. High Throughput Localized Passive Diffusion Device for Patterning Cuadal-Rostral Hind Brain Neuro-Organoids

### Induction of Rostral-Caudal Patterned Brain Organoid by a Retinoic Acid Activation Gradient

In vivo, the human brain and center nerve system are patterned in the rostral-caudal direction with the forebrain, midbrain, hindbrain, and spinal cord, which is partially dictated by a gradient of RA, WNT signaling, and FGF8 proteins. To generate rostral-caudal patterned organoids, the neural differentiation protocol provided above was modified by introducing RA as the morphogen. The SMAD inhibition and neural induction parts are the same as the D-V patterning protocol. 100 μM retinoic acid was supplemented from day 14 to day 22 in the morphogen chamber for hindbrain induction. The organoid was released from the device 500 on day 23.

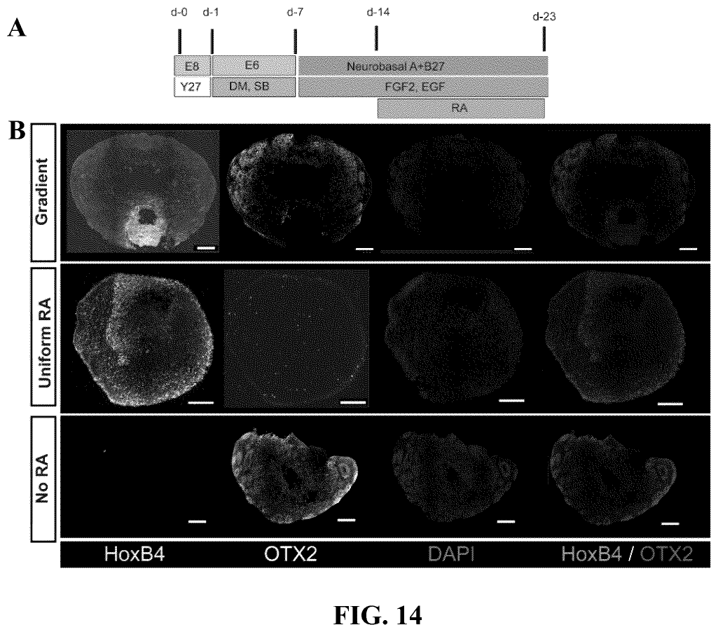

It was established that a low concentration of RA could facilitate and induction of the hindbrain organoid. By modifying the neural induction protocol, a R-C patterned brain organoid was generated with 23 days of differentiation. The protocol was started with dual-SMAD inhibition, followed by neural induction with the neurobasal medium. Due to the instability of RA, a higher concentration (100 μM) of RA was added to the gradient chamber from day 14 to day 22. As shown in FIG. 14, in the gradient group, HoxB4+ was observed on the bottom region of the organoid, indicating hindbrain expression. Furthermore, FIG. 14 shows that OTX2+ was observed in the top part of the organoid, indicating forebrain expression. By adding RA to the medium, a uniform expression of HoxB4 was observed. Without RA, the organoid will express OTX2. Therefore, the device 500 can generate an R-C patterned organoid with a gradient of RA.

### Discussion of Examples

The devices and methods provided herein allow for precise control of a morphogenic gradient through simple gel-based diffusion. In one aspect, disclosed herein is a high-throughput, reliable, control-release diffusion device to pattern hPSC-derived organoids into dorsal-ventral (“D-V”) and rostral-caudal (“R-C”) patternings. In some examples, the devices provided herein are high-throughput systems configured to mimic the SHH and RA gradient presented in-vivo for dorsal-ventral and rostral-caudal patternings, respectively. Using these methods and techniques, D-V and/or R-C patterned brain organoids were successfully generated. Long-term culturing of the D-V patterned organoid revealed active neurogenesis and astrogenesis.

Several disadvantages in conventional methods of 3D organoid differentiation and gradient generation can be overcome by the present disclosure. Protocols for brain organoids differentiation are prone to variable results and can be expensive for long-term culturing. Among many other advantages, the devices provided herein are easy to operate and highly reliable. In one example using the presently disclosed systems and methods, six organoids were cultured in separated medium chambers, and only 2 ml medium was required per day. Thus, the devices and methods provided herein are inexpensive, easy to fabricate, and easy to operate.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

What is claimed is:

1. A device for patterning organoids comprising:
a plurality of layers vertically arranged, the plurality of layers comprising:
a base layer;
a diffusion medium disposed on the base layer;
a microwell layer disposed above the diffusion medium, the microwell layer comprising a lower surface, an upper surface, and defining one or more microwells, each of the one or more microwells defining an opening extending between the respective microwell and the diffusion medium; and
a partitioning layer disposed above the diffusion medium, the partitioning layer comprising a wall that defines a chemical reservoir and one or more medium chambers, wherein the wall divides the chemical reservoir from the one or more medium chambers.

2. The device of claim 1, wherein cells are disposed within the one or more microwells.

3. The device of claim 2, wherein one or more morphogens are disposed within the chemical reservoir, wherein diffusion of the one or more morphogens from the lower surface to the upper surface creates a morphogenic gradient within each of the one or more microwells.

4. The device of claim 3, wherein the cells comprise cells of a first type and cells of a second type.

5. The device of claim 4, wherein the one or more morphogens differentiate the cells of a first type and the cells of a second type into an organoid disposed within the microwells.

6. The device of claim 4, wherein cells of the first type and cells of the second type are differentially disposed along a vertically-oriented differentiation axis according to the morphogenic gradient, wherein the arrangement of the first type and the second type of cells defines a patterning.

7. The device of claim 1, wherein the medium chambers are radially outward from the chemical reservoir.

8. The device of claim 7, wherein each medium chamber contains a microwell.

9. The device of claim 1, wherein the device is sized and configured to fit within a standard six-well plate.

10. The device of claim 1, wherein the microwell layer comprises an inorganic polymer.

11. The device of claim 1, wherein the diffusion medium comprises cross-linked gelatin.

12. The device of claim 1, wherein the device further comprises one or more alignment structures configured to maintain the relative orientation of the plurality of layers.

13. The device of claim 12, wherein the one or more alignment structures comprise a suture.

14. The device of claim 12, wherein the device further comprises an anchoring layer coupled to the base layer, the anchoring layer defining one or more openings configured to receive the one or more alignment structures, thereby anchoring the one or more alignment structures to the base layer.

15. A method for producing a patterned organoid, the method comprising:
seeding one or more microwells defined in a microwell layer with a plurality of cells to generate a cell aggregate;
adding one or more morphogens to a chemical reservoir defined by a partitioning layer, the partitioning layer comprising a wall that divides the chemical reservoir from one or more medium chambers, wherein the one or more microwells are defined within the one or more medium chambers;
diffusing the one or more morphogens through a diffusion medium disposed beneath the microwell layer, wherein the diffusion medium is in fluid communication with the chemical reservoir;
releasing the one or more morphogens through openings defined at respective base regions of the one or more microwells, wherein each respective opening extends between the microwell and the diffusion medium;
forming a morphogenic gradient within the one or more microwells along a vertically-oriented differentiation axis defined by each of the one or more microwells; and
obtaining a patterned organoid from the cell aggregate comprised of cells differentiated according to the morphogenic gradient.

16. The method of claim 15, wherein the patterned organoid is a neuro-organoid.

17. The method of claim 15, wherein the one or more morphogens comprise a sonic hedgehog agonist.

18. The method of claim 16, wherein the patterned organoid is a dorsal-ventral patterned forebrain organoid.

19. The method of claim 15, wherein the one or more morphogens comprise retinoic acid.

20. The method of claim 16, wherein the patterned organoid is a rostral-caudal patterned hindbrain organoid.
